(19) United States
(12) Patent Application Publication
(10) Pub. No.: US 2022/0175769 A1
(43) Pub. Date: Jun. 9, 2022

(54) METHOD OF TREATMENT OF ACTINIC KERATOSES

(71) Applicant: Ankh Life Sciences Limited, Dublin 2 (IE)

(72) Inventors: Gene H. Zaid, Hutchinson, KS (US); Cameron E. West, Sterling, KS (US); Robert Preston Moore, Great Bend, KS (US); Paul Dent, Glen Allen, VA (US); Shawn G. Kwatra, Baltimore, MD (US)

(73) Assignee: Ankh Life Sciences Limited, Dubline 2 (IE)

(21) Appl. No.: 17/115,633

(22) Filed: Dec. 8, 2020

(51) Int. Cl. A61K 31/4965; A61K 31/437; A61K 31/11; A61K 36/9066; A61K 9/00; A61K 31/085

(52) U.S. Cl. CPC A61K 31/4965; A61K 31/437; A61K 31/085; A61K 36/9066; A61K 9/0014; A61K 31/11

(57) ABSTRACT

Novel medicaments for the treatment of actinic keratoses are provided, along with methods of treatment. The medicaments include respective quantities of curcumin, harmine, and isovanillin, which can be used alone or in combination with fluorouracil. In the latter cases, the three-component compositions provide a synergistic or adjuvant effect with fluorouracil.

### BACKGROUND OF THE INVENTION

### Field of the Invention

The present invention is broadly concerned with medicaments and methods for the treatment of actinic keratoses. More particularly, it is concerned with such medicaments and methods wherein the medicaments include curcumin, harmine, and isovanillin, and preferably further include fluorouracil (5FU). The four-component medicament provides improved results, owing to synergistic or adjuvant effectiveness.

### Description of the Prior Art

Actinic keratosis (AK) is a common skin condition in the form of precancerous skin growths resulting from overexposure to the sun's harmful rays, and often appears as reddish spots on the face, ears, balding scalp, hands, neck, or lips. Dermatologists diagnose AK by a simple skin examination, or a skin biopsy. It is often impossible to tell which AK patches or lesions develop into skin cancer (keratinocyte carcinoma) and, accordingly, these are usually removed as a precautionary measure.

A number of medications have been employed for the treatment of AK. These include 5FU (fluorouracil), Imiquimod, Ingenol, and Diclofenic, in the form of creams or gels. Generally, 5FU is considered to be the treatment of choice.

GZ17-6.02 is an oral synthetic investigational compound that is a mixture of three originally plant-derived components, namely curcumin, harmine, and isovanillin. GZ17-6.02 is currently undergoing Phase I oncology trials in the USA, after having demonstrated in vivo activity against pancreatic cancer, colorectal cancer, and head and neck squamous cell carcinoma. This product is described in a variety of patents, including U.S. Pat. No. 9,402,834; this patent is incorporated by reference herein in its entirety.

### SUMMARY OF THE INVENTION

The present invention provides improved medicaments for the treatment of actinic keratoses, as well as methods of use thereof. In one aspect of the invention, the new medicaments comprise or consist essentially, or even consist of, the combination of curcumin, harmine, isovanillin, and fluorouracil.

Curcumin (diferuloylmethane, 1,7-bis(4-hydroxy3-mcthoxyphenyl)-1,6-heptadiene-3,5-dione) is a symmetrical diphenolic dienone, see structure C-1 below. It exists in solution as an equilibrium mixture of the symmetrical dienone (diketo) and the keto-enol tautomer; the keto-enol form is strongly favored by intramolecular hydrogen bonding.

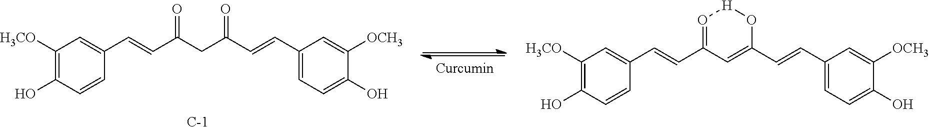

Curcumin contains two aryl rings separated by an unsaturated 7-carbon linker having a symmetrical β-diketone group (as used herein, “β-diketone” embraces both tautomeric forms, namely the diketo and enol forms). The aryl rings of curcumin contain a hydroxyl group in the para position and a methoxy group in the meta position.

The chemical structure of harmine, 1-methyl-7-methoxy-β-carboline, is shown as follows:

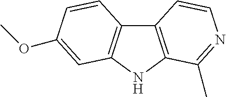 [SMILES: COc1ccc2c(c1)[nH]c1c(C)nccc12]

Isovanillin is a phenolic aldehyde having a hydroxyl group at the meta position and a methoxy group at the para position. Isovanillin is illustrated in the following structure:

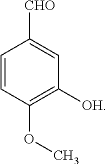

In such medicaments, the curcumin is present at a level of from about 5-40% by weight, the harmine is present at a level of from about 7-50% by weight, the isovanillin is present at a level of from about 25-85% by weight, and the fluorouracil is present at a level of from about 0.5-20 by weight, all of the foregoing based upon the total weight of the curcumin, harmine, isovanillin, and fluorouracil taken as 100% by weight. In many instances, particularly when topical medicaments are desired, the curcumin, harmine, isovanillin, and fluorouracil are dispersed in a non-interfering solvent, such as solvents selected from the group consisting of C1-C4 alcohols, DMSO, and mixtures thereof. The medicament may include additional inactive pharmaceutically-acceptable ingredients and/or vehicles as a base carrier composition in which the active ingredients are dispersed. As used herein, the term “pharmaceutically-acceptable” means not biologically or otherwise undesirable, in that it can be administered to a subject without excessive toxicity, irritation, or allergic response, and does not cause any undesirable biological effects or interact in a deleterious manner with any of the other components of the composition in which it is contained. The term “carrier,” as used herein, means one or more compatible base compositions with which the active ingredient (e.g., curcumin, harmine, isovanillin, and optional fluorouracil) is combined to facilitate the administration of ingredient, and which is suitable for administration to a patient. Such preparations may also routinely contain salts, buffering agents, preservatives, and optionally other therapeutic ingredients. The carrier would naturally be selected to minimize any degradation of the active ingredient and to minimize any adverse side effects in the subject, as would be well known to one of ordinary skill in the art. Pharmaceutically-acceptable ingredients include those acceptable for veterinary use as well as human pharmaceutical use. Exemplary carriers include petrolatum, mineral oil, alcohols (e.g., cetyl alcohol, stearyl alcohol), propylene glycol, nonionic surfactants and/or emulsifiers (e.g., polysorbates), polymers, parabens, silicones, waxes, preservatives, aqueous solutions, and combinations thereof.

In this aspect, methods of treating actinic keratosis comprise the step of contacting actinic keratosis cells with the aforementioned medicament. In most instances, such contacting will be affected by topical application of the medicament in a liquid or flowable form (e.g., ointments, creams, pastes, lotions, or gels), although systemic administration of the medicament is a possibility, e.g., a patient suffering from actinic dermatosis receives the medicament by any suitable route.

It has also been discovered that the three-component mixtures of the invention, such as the 6.02 composition, is effective when used in the absence of fluorouracil. Thus, in this second aspect of the invention, a method of treating actinic keratosis is provided which comprises the step of contacting actinic keratosis cells with a medicament comprising curcumin, harmine, and isovanillin, or by treatment of a human patient suffering from actinic keratosis by administering the three-component composition. Such a three-component composition would typically have a ratio of isovanillin:harmine:curcumin of approximately 0.1-25:0.1-5:0.1-5. In terms of amounts, the isovanillin being is at a level of from about 25-85% by weight, the harmine is present at a level of from about 7-50% by weight, and the curcumin being is at a level of from about 5-40% by weight, all based on the total weight of the isovanillin, harmine, and curcumin taken as 100% by weight. Moreover, in certain embodiments the isovanillin is present at a level of at least about three times greater than each of the harmine and curcumin.

Thus, while an effective medicament for the treatment of actinic keratosis may be limited to the three-member curcumin/harmine/isovanillin compositions, these compositions provide a synergistic and/or adjuvant effect when used in combination with fluorouracil.

### BRIEF DESCRIPTION OF THE DRAWINGS

FIG. 1 is a graph comparing the interaction of GZ17-6.02 with (A) 5FU and with (B) HDAC inhibitors to kill AK cells (n=3+/−SD).

FIGS. 2-21 are graphs illustrating the effect of GZ17-6.02 (“602”), 5-fluorouracil (“5FU”), and/or the drugs in combination (“602+5FU”), on expression levels and activity/phosphorylation after 6 h in AK cells. The graphical data presented are the normalized amount of fluorescence set at 100% comparing intensity values for vehicle control (n=3+/−SD). More specifically:

FIG. 2 is a graph showing the data for ATM expression levels (ATM) and activity/phosphorylation (P-ATM).

FIG. 3 is a graph showing the data for AMPK expression levels (AMPK) and activity/phosphorylation (P-AMPK).

FIG. 4 is a graph showing the data for different UKL1 expression levels and activity/phosphorylation (P-ULK1).

FIG. 5 is a graph showing the data for mTOR expression levels and activity/phosphorylation (P-mTOR).

FIG. 6 is a graph showing the data for ERK 1/2 expression levels and activity/phosphorylation (P-ERK 1/2).

FIG. 7 is a graph showing the data for AKT expression levels and activity/phosphorylation (P-AKT).

FIG. 8 is a graph showing the data for K-RAS and N-RAS expression levels;

FIG. 9 is a graph showing the data for PERK expression levels and activity/phosphorylation (P-PERK).

FIG. 10 is a graph showing the data for eLF2α expression levels and activity/phosphorylation (P-eLF2α).

FIG. 11 is a graph showing the data for Beclin1 and ATG5 expression levels.

FIG. 12 is a graph showing the data for cathepsin B and AIF expression levels.

FIG. 13 is a graph showing the data for YAP expression levels and activity/phosphorylation (P-YAP).

FIG. 14 is a graph showing the data for TAZ expression levels and activity/phosphorylation (P-TAZ).

FIG. 15 is a graph showing the data for CD95 and FADD expression levels.

FIG. 16 is a graph showing the data for various ERBB expression levels.

FIG. 17 is a graph showing the expression levels for c-KIT and c-MET.

FIG. 18 is a graph showing the expression levels for HSP90, HSP70, and GRP78 chaperones.

FIG. 19 is a graph showing the data for p70 S6K expression levels and activity/phosphorylation (P-p70 S6K).

FIG. 20 is a graph showing the data for MEK1 expression levels and activity/phosphorylation (P-MEK1).

FIG. 21 is a graph showing the expression levels for HDAC6 and HDAC11.

FIG. 22 is a set of images of stains at 60× magnification, showing expression of K-RAS and N-RAS.

FIG. 23 is a graph of the effect on cell death after 24 hours of exposure to vehicle control (“veh”), GZ17-6.02 (“602”), 5-fluorouracil (“5fu”), and or the drugs in combination (“602 5fu”) in cells with knockdown of various proteins as indicated. * p<0.05 less than corresponding value in siSCR cells; ** p<0.05 less than corresponding values in siATM and siAMPKα cells.

FIG. 24A is a graph of the effect on cell death after 24 hours of exposure to vehicle control (“veh”), GZ17-6.02 (“602”), 5-fluorouracil (“5fu”), and or the drugs in combination (“602 5fu”) in cells transfected with an empty control vector plasmid (“CMV”) or with plasmids to express the indicated proteins. ** p<0.05 less than corresponding values in siATM and siAMPKα/ca MEK1 and ca STAT3 cells.

FIG. 24B is a graph of the effect on cell death after 24 hours of exposure to vehicle control (“veh”), GZ17-6.02 (“602”), 5-fluorouracil (“5fu”), and or the drugs in combination (“602 5fu”) in cells transfected with an empty control vector plasmid (“CMV”) or with plasmids to express the indicated proteins. ** p<0.05 less than corresponding values in ca MEK1 and ca STAT3 cells.

FIG. 25A is a graph of autophagosome formation in cells transfected with an activated mTOR mutant protein and exposed to vehicle control (“veh”), GZ17-6.02 (“602”), 5-fluorouracil (“5fu”), and or the drugs in combination (“602 5fu”).

FIG. 25B is a graph of cell death in cells transfected with an activated mTOR mutant protein and exposed to vehicle control (“veh”), GZ17-6.02 (“602”), 5-fluorouracil (“5fu”), and or the drugs in combination (“602 5fu”).

FIG. 26 is a graph of the effect on cell death after exposure to vehicle control (“veh”), GZ17-6.02 (“602”), 5-fluorouracil (“5fu”), and or the drugs in combination (“602 5fu”) in cells with knockdown of various proteins as indicated.

### DETAILED DESCRIPTION OF THE PREFERRED EMBODIMENT

The present invention provides new methods for the treatment of AK. In one aspect of the invention, a treatment composition made of curcumin, harmine, and isovanillin is employed (generally referred to as GZ17-6.02), and in another aspect this treatment composition is combined with 5FU to give still greater treatment efficacy. Methods described herein include methods for modulating autophagy in treated AK cells using the medicaments, and in particular methods for inducing or activating autophagy in treated cells, increasing AK cell death in treated cells, and promoting apoptosis and formation of autophagosomes.

### GZ17-6.02

This composition is prepared by combining individual quantities of normally highly purified curcumin, harmine, and isovanillin components at ratios of approximately 0.1-25:0.1-5:0.1-5 (isovanillin:harmine:curcumin). Each such component may be made up of one or more isovanillin, harmine, and/or curcumin compounds. Generally, it is preferred that the isovanillin component is the preponderant component in the composition on a weight basis, with the harmine and curcumin components being present in lesser amounts on a weight basis. Still further, the isovanillin component may be present at a level of at least three times (more preferably at least five times) greater than that of each of the harmine and curcumin components. In terms of amounts of the three components, the isovanillin component should be present at a level of from about 25-85% by weight, the harmine component should be present at a level of from about 7-50% by weight, and the curcumin component should be present at a level of from about 5-40% by weight, all based on the total weight of the three components taken as 100% by weight.

The single most preferred GZ17-6.02 product, and that tested in the examples, was made by dispersing quantities of solid synthetic isovanillin (771 mg, 98% by weight purity), synthetic harmine (130.3 mg, 99% by weight purity), and a commercially available curcumin product derived by the treatment of turmeric (98.7 mg, containing 99.76% by weight curcuminoids, namely 71.38% curcumin, 15.68% demethoxycurcumin, and 12.70% bisdemethoxycurcumin), in a 1 mL ethanol at a weight ratio of 771:130.3:98.7 (isovanillin:harmine:curcumin product) in ethanol followed by sonication of the dispersion.

As used herein, “curcumin,” “harmine,” and “isovanillin” means, respectively, curcumin, harmine, and isovanillin, and the isomers, tautomers, enantiomers, esters, derivatives, metal complexes (e.g., Cu, Fe, Zn, Pt, V), prodrugs, solvates, metabolites, and pharmaceutically acceptable salts of any of the foregoing.

“Isomers” refers to each of two or more compounds with the same formula but with at different arrangement of atoms, and includes structural isomers and stereoisomers (e.g., geometric isomers and enantiomers); “tautomers” refers to two or more isometric compounds that exist in equilibrium, such as keto-enol and imine and enamine tautomers; “derivatives” refers to compounds that can be imagined to arise or actually be synthesized from a defined parent compound by replacement of one atom with another atom or a group of atoms; “solvates” refers to interaction with a defined compound with a solvent to form a stabilized solute species; “metabolites” refers to a defined compound which has been metabolized in vivo by digestion or other bodily chemical processes; and “prodrugs” refers to defined compound which has been generated by a metabolic process. The compounds can be directly used in partial or essentially completely purified forms, or can be modified as indicated above. The compounds may be in crystalline or amorphous forms, and may be lyophilized.

“Pharmaceutically acceptable salts” with reference to the components means salts of the components which are pharmaceutically acceptable, i.e., salts which are useful in preparing pharmaceutical compositions that are generally safe, non-toxic, and neither biologically nor otherwise undesirable and are acceptable for human pharmaceutical use, and which possess the desired degree of pharmacological activity. Such pharmaceutically acceptable salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts Properties, and Use, P. H. Stahl & C. G. Wermuth eds., ISBN 978-3-90639-058-1 (2008).

### EXAMPLE

The following examples set forth methods in accordance with the invention. It is to be understood, however, that these examples are provided by way of illustration and nothing therein should be taken as a limitation upon the overall scope of the invention.

Materials

5FU was purchased from Selleckchem (Houston, Tex.). GZ17-6.02 (curcumin (2.0 μM)+harmine (4.5 μM)+isovanillin (37.2 μM) in DMSO) was supplied by Genzada Pharmaceuticals LLC (Sterling, KS). AK cells (HT297.T) were obtained from the ATCC (Bethesda, Md.) and were not further validated beyond that provided by the vendor. Control studies were also carried out to verify on-target specificity of antibodies to detect total protein levels and phosphorylated levels of proteins (not shown).

Methods

Culture, Viability, Transfection and in vitro exposure of cells to drugs. The AK cells were plated on 96-well plates (cell density ˜5,000/well) and cultured at 37° C. (5% (v/v CO₂) in vitro using RPMI supplemented with 5% (v/v) fetal calf serum and 10% (v/v) non-essential amino acids for 24 hours before drug exposure.

AK cells, as indicated, were treated with vehicle control, GZ17-6.02 [curcumin (2.0 μM)+harmine (4.5 μM)+isovanillin (37.2 μM)], 5FU (50 μM), vorinostat (250 nM), entinostat (50 nM) or in combination. Cells were isolated 24 h afterwards and viability determined via trypan blue exclusion assays (n=3+/−SD).

AK cells were transfected with a scrambled siRNA or with siRNA molecules to knock down expression of the indicated proteins, or with a control empty vector plasmid (CMV) or plasmids to express the indicated proteins. Twenty-four hours later, cells were treated with vehicle control, GZ17-6.02 (final curcumin concentration 2.0 μM), 5FU (50 μM) or the drugs in combination for 24 h. Cells were isolated and viability determined via trypan blue exclusion assays (n=3+/−SD).

Detection of protein expression and protein phosphorylation by in-cell western blotting using a Hermes WiScan microscope. Cells were treated with vehicle control, GZ17-6.02 (final curcumin concentration 2.0 μM), 5FU (50 μM) or the drugs in combination for 6 h. At various time-points after the initiation of drug exposure, cells were fixed in situ, permeabilized, and stained with the indicated validated primary antibodies and imaged with secondary antibodies carrying red and green fluorescent tags. The staining intensity of at least 100 cells per well/condition was determined in three separate studies. Cells were visualized at either 10× magnification for bulk assessments of immunofluorescent staining intensity or at 60× magnification for assessments of protein or protein-protein colocalization. Total protein expression was assessed along with changes in protein activity based upon phosphorylation profiles. Fluorescence intensity was normalized to the vehicle control set at 100% as the baseline for comparison to the treated cells (n=3+/−SD).

Data analysis. Analyses used one-way ANOVA and a two tailed Student's t-test. Differences with a p-value of <0.05 were considered statistically significant. Experiments are the means of multiple individual points from multiple experiments (±SD).

Results

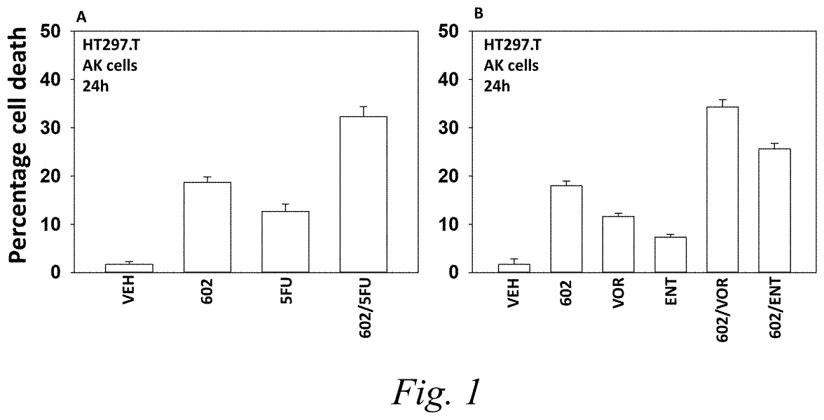

A topical formulation of GZ17-6.02 was tested to determine whether GZ17-6.02 could kill epidermal actinic keratosis cells and whether it interacted with a standard of care therapeutic, 5FU, to cause increasing amounts of cell death. GZ17-6.02 and 5FU interacted in an additive fashion to kill AK cells (FIG. 1A). Histone deacetylase inhibitors also enhanced GZ17-6.02 lethality in AK cells (FIG. 1B). As a single agent, GZ17-6.02 caused approximately 20% cell death in the AK cells in 24 h.

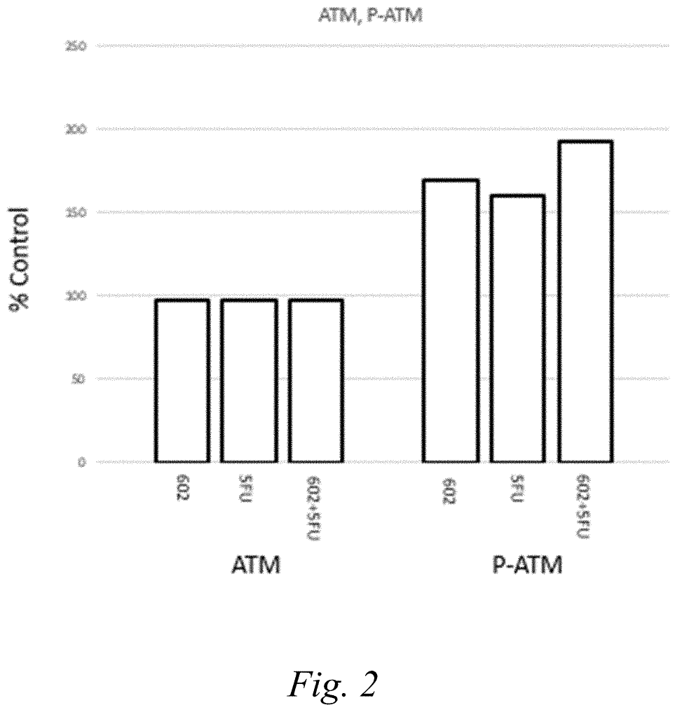

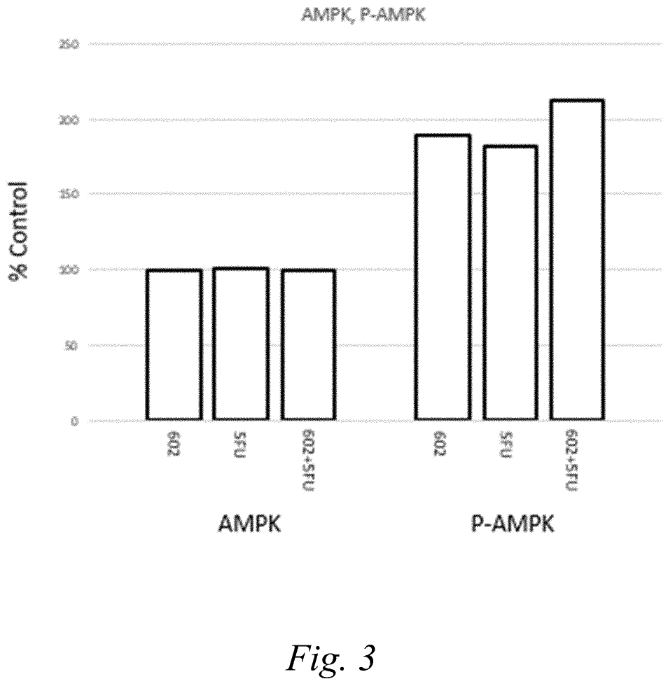

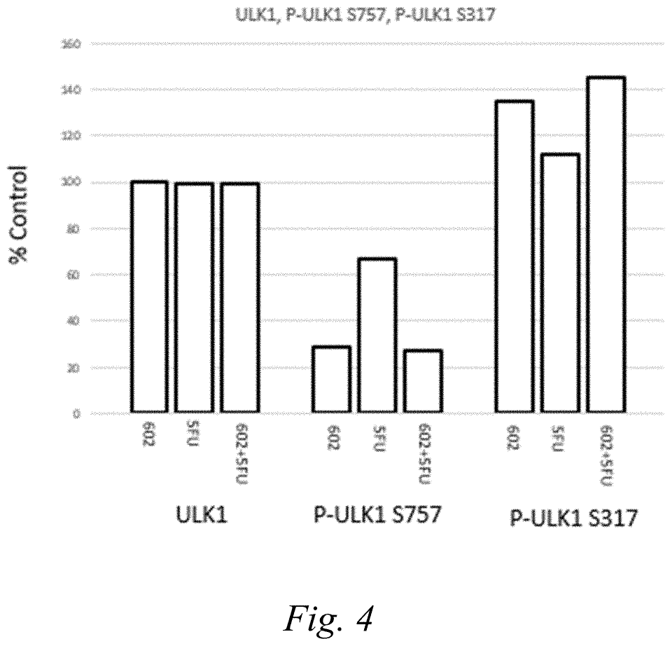

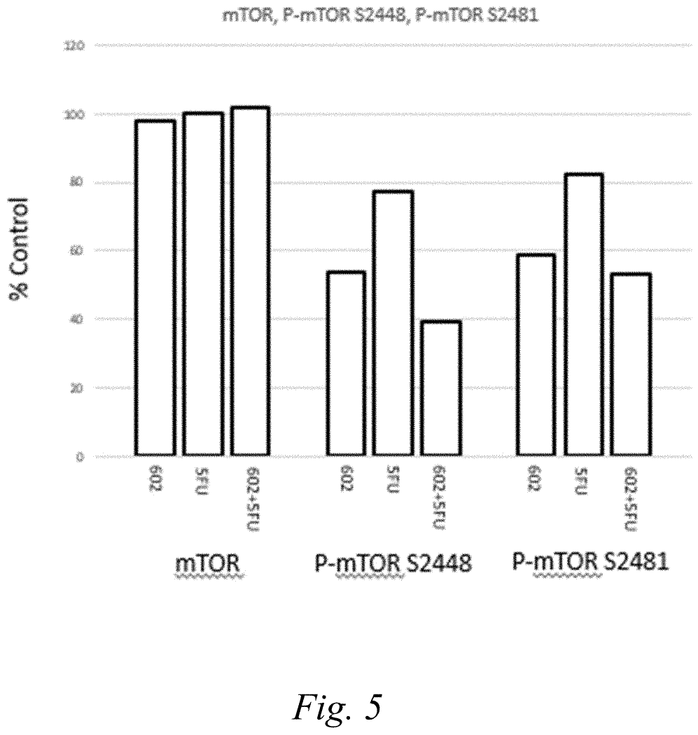

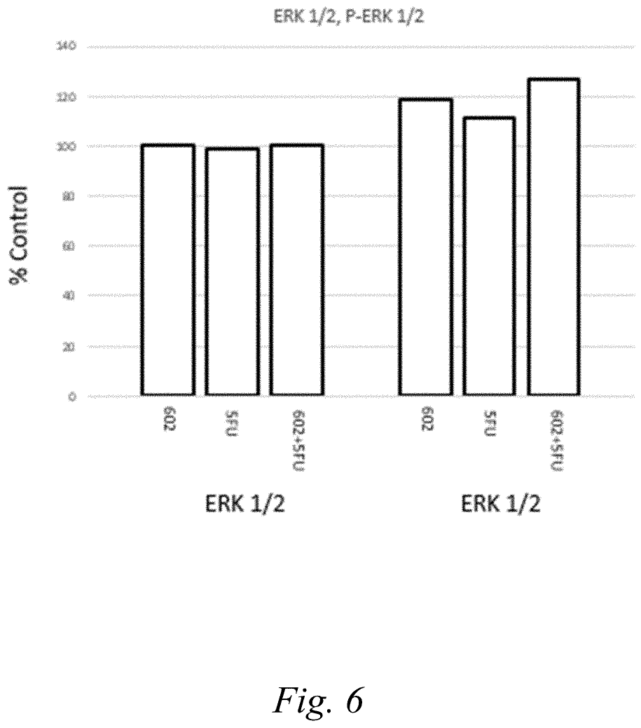

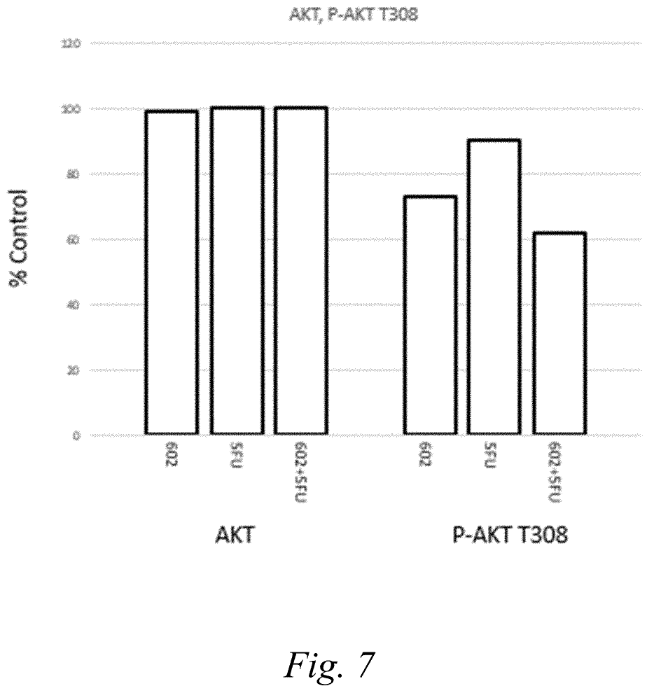

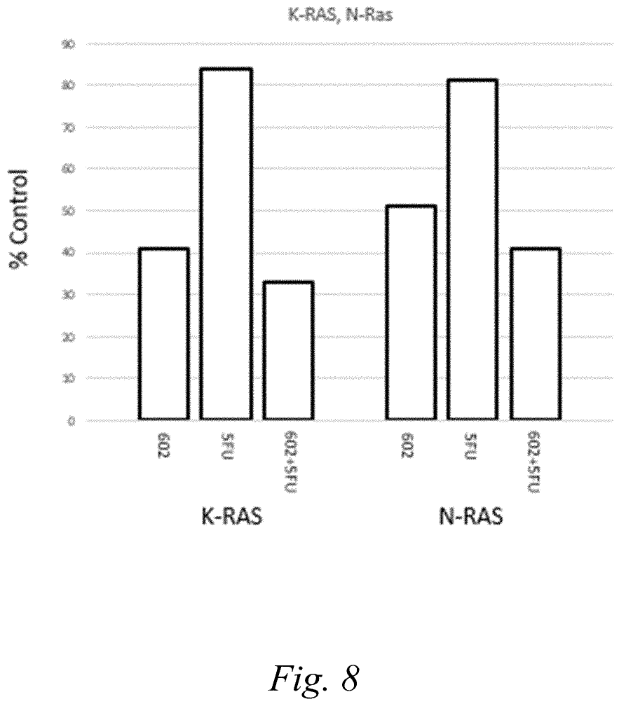

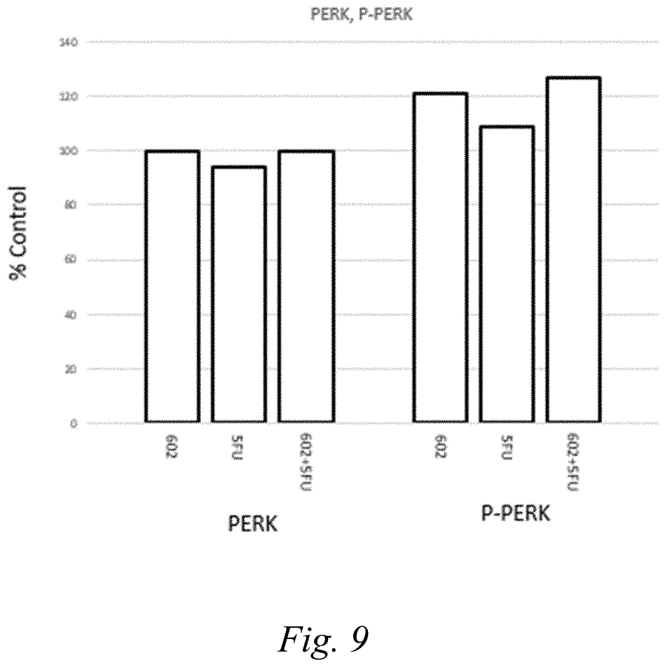

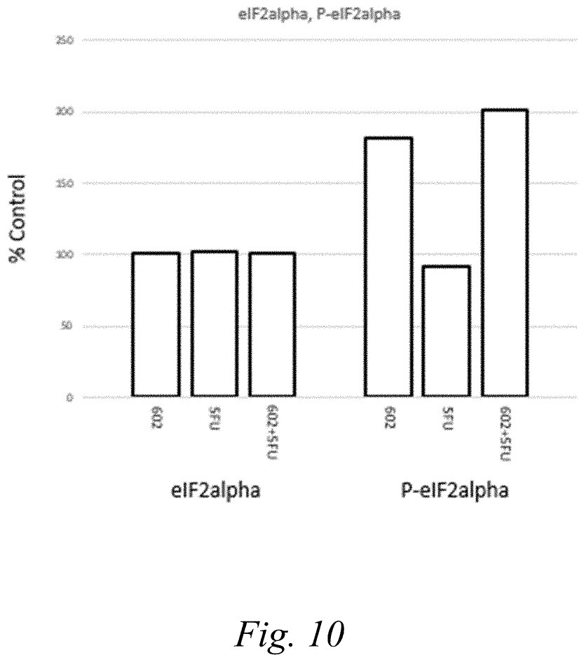

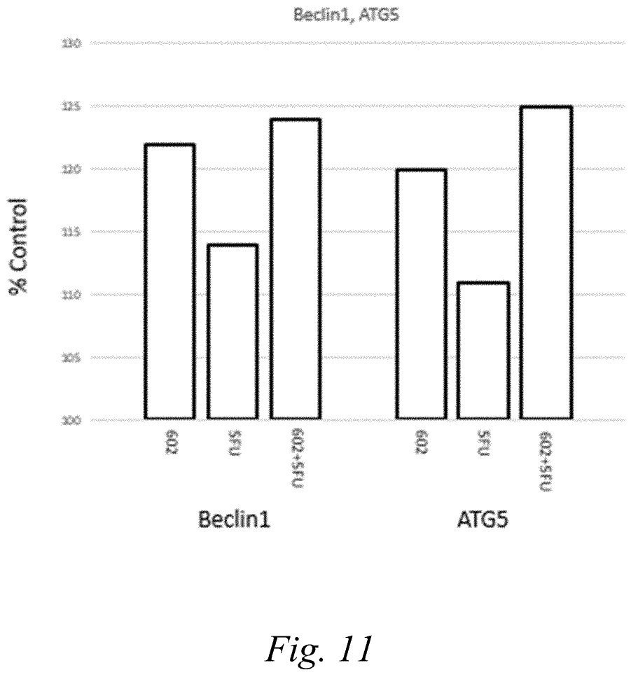

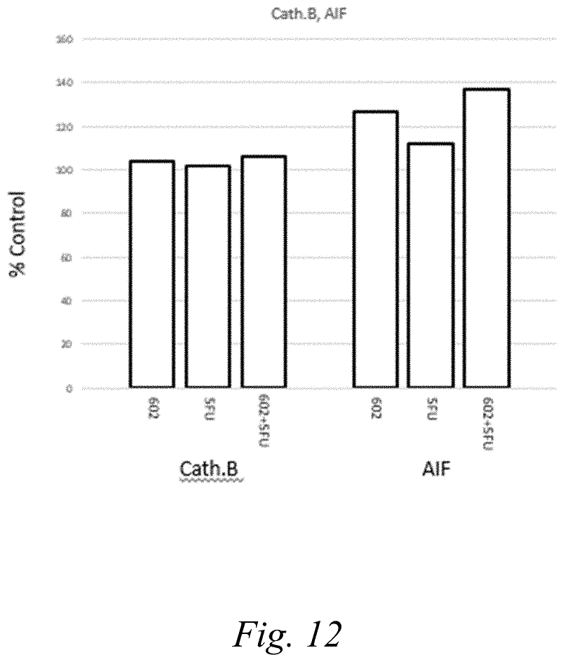

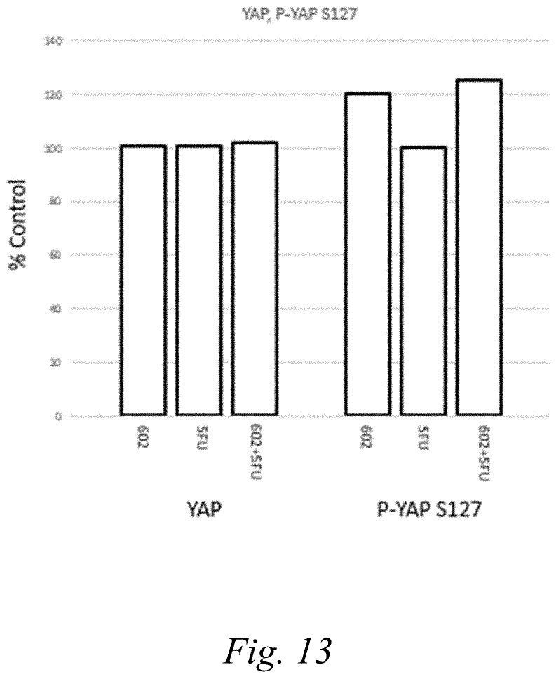

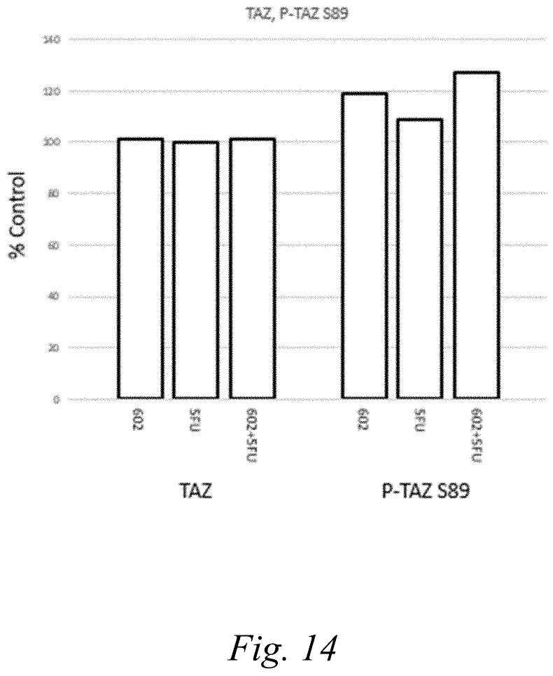

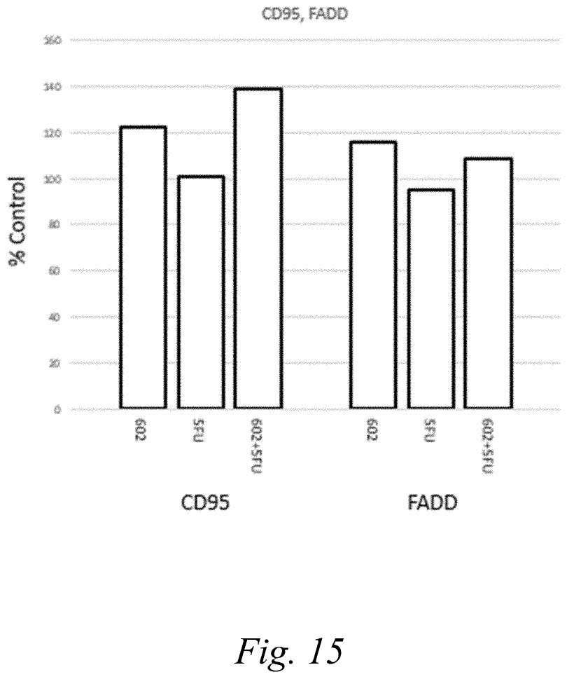

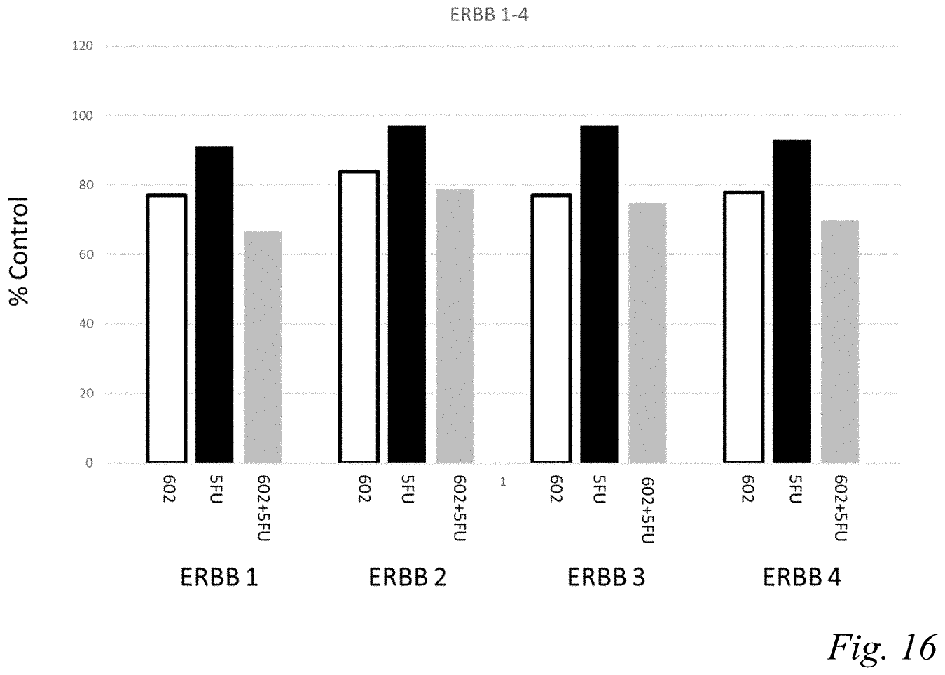

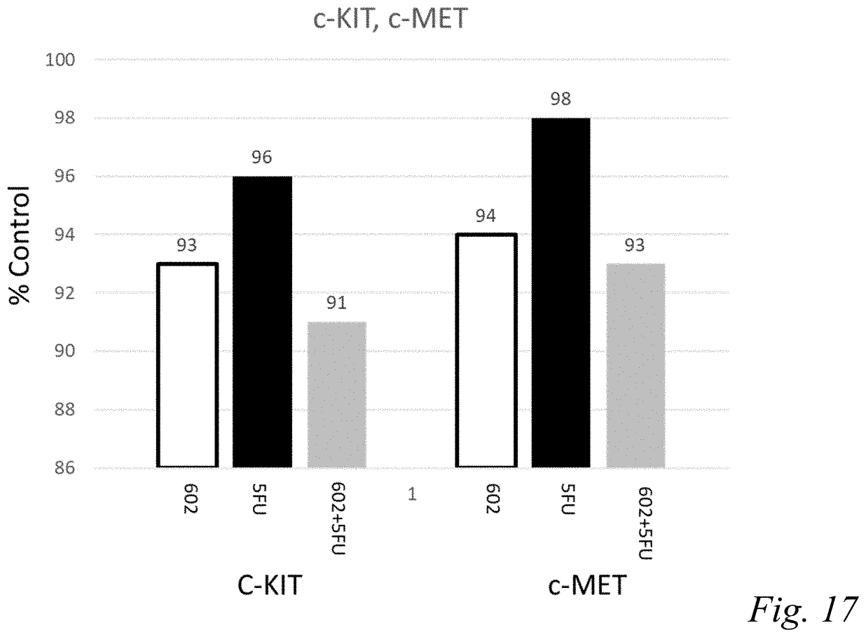

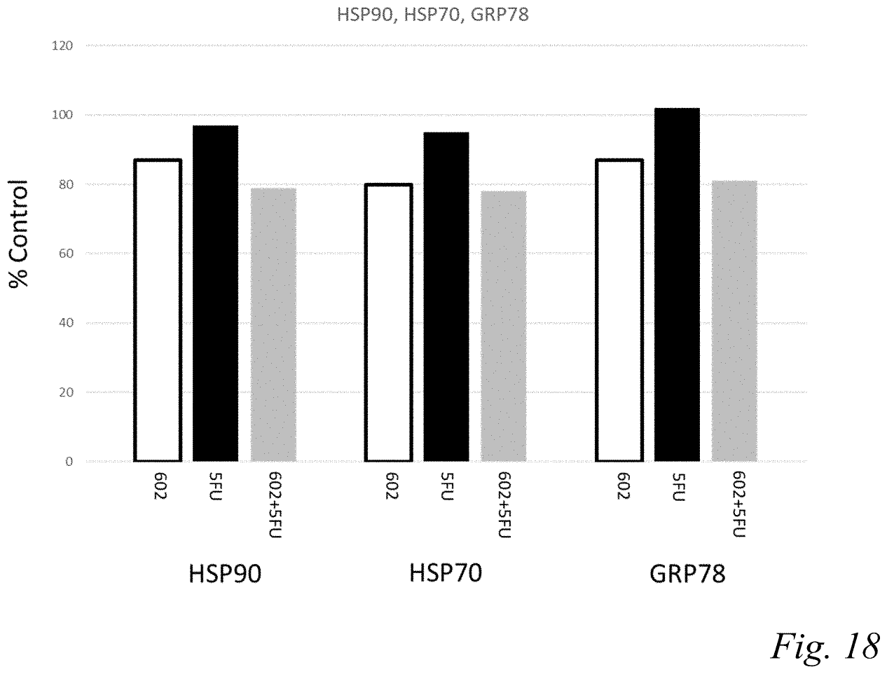

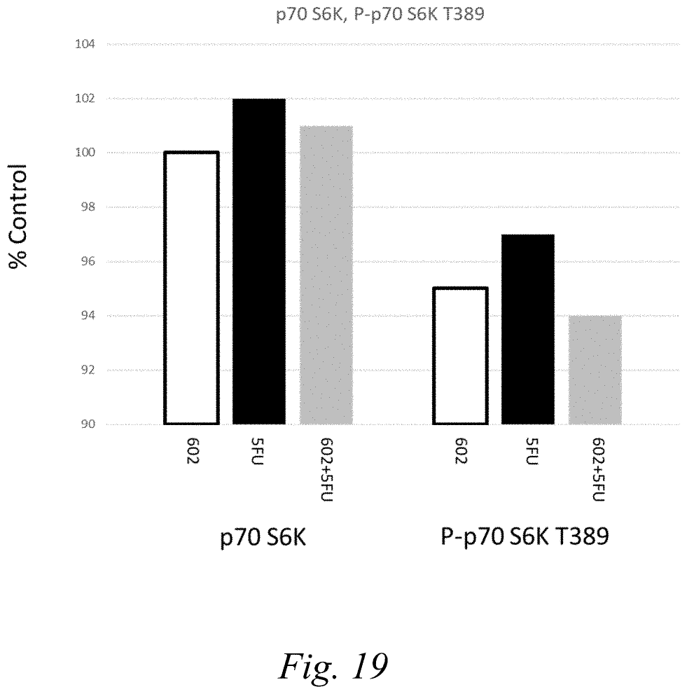

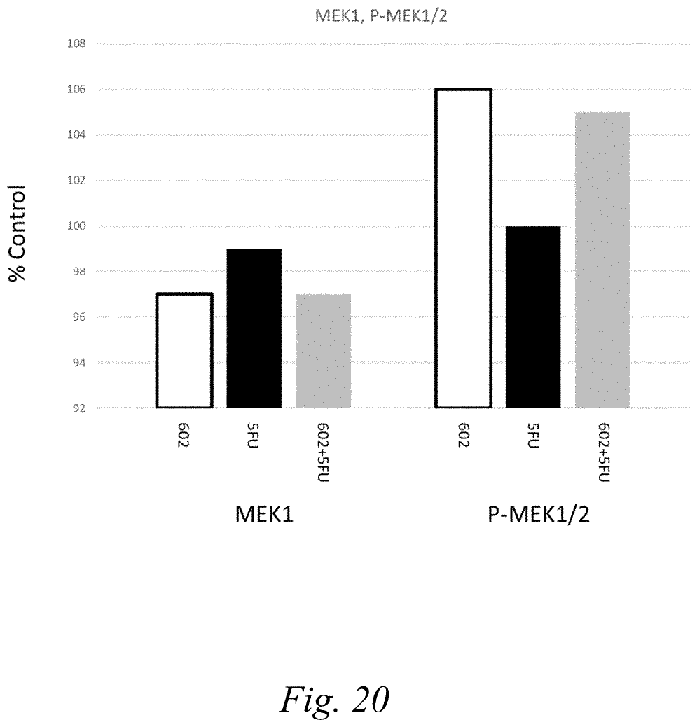

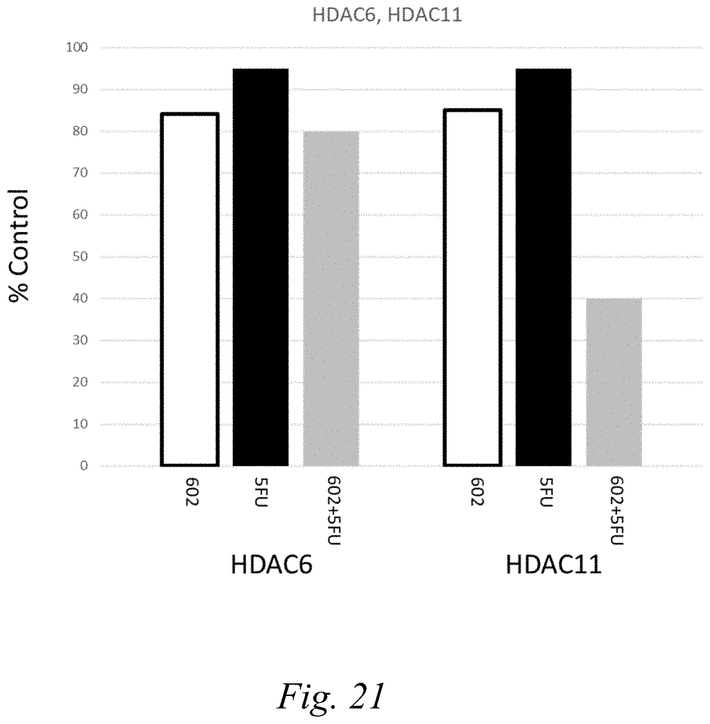

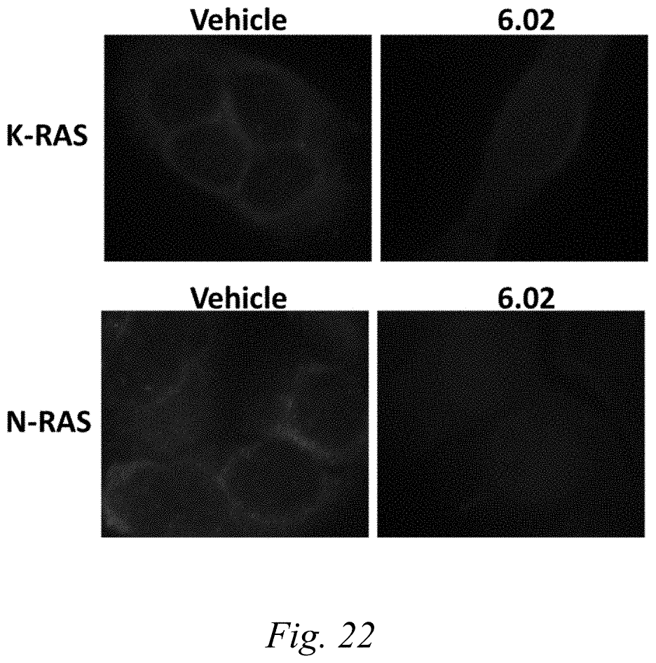

In the recent GZ17-6.02 studies published by Booth et al. (GZ17-6.02 initiates DNA damage causing autophagosome-dependent HDAC degradation resulting in enhanced anti-PD1 checkpoint inhibitory antibody efficacy. J Cell Physiol. (2020)), we performed wide ranging agnostic screening analyses to monitor changes in expression/function of multiple signal transduction pathways whose biology we had previously linked in multiple prior manuscripts to chemotherapy biology. First, we assessed the effect of different treatments or vehicle control on protein expression level and activity (based upon phosphorylation) in AK cells. The data are shown in FIGS. 2-21. In the transformed AK cell line GZ17-6.02, as a single agent, activated ATM (FIG. 2), the AMPK (FIG. 3), ULK1 (FIG. 4), ERK1/2 (FIG. 6) and inactivated mTORC1 (FIG. 5), mTORC2 (FIG. 5), AKT (FIG. 7), eIF2α (FIG. 10), YAP (FIG. 13; Hippo pathway) and TAZ (FIG. 14). These events predict for enhanced autophagosome formation, reduced protein translation and tumor cell invasion and enhanced susceptibility to undergoing death processes. GZ17-6.02 and 5FU interacted to reduce the expression of ERBB1 and ERBB4 (FIG. 16), RAS proteins (FIG. 8), and HDAC6 and HDAC11 (FIG. 21) and to enhance the phosphorylation (activity) of ATM and AMPKα (FIGS. 2-3). The drug combination further reduced the phosphorylation of mTORC1 (FIG. 5) and increased the expression of CD95 (FIG. 15). This data predicts for greater levels of autophagosome formation and for signaling by the extrinsic apoptosis pathway. As judged based on fluorescence intensity and pictorially, GZ17-6.02 reduced the expression of K-RAS and N-RAS (FIG. 22). Collectively our signaling data predict for significant levels of autophagosome formation, reduced expression of multiple proteins and for elevated death receptor signaling.

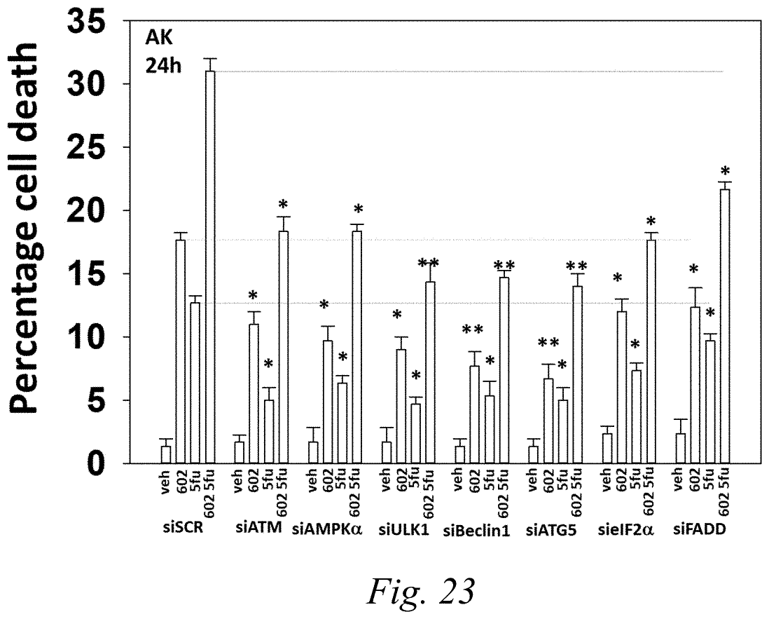

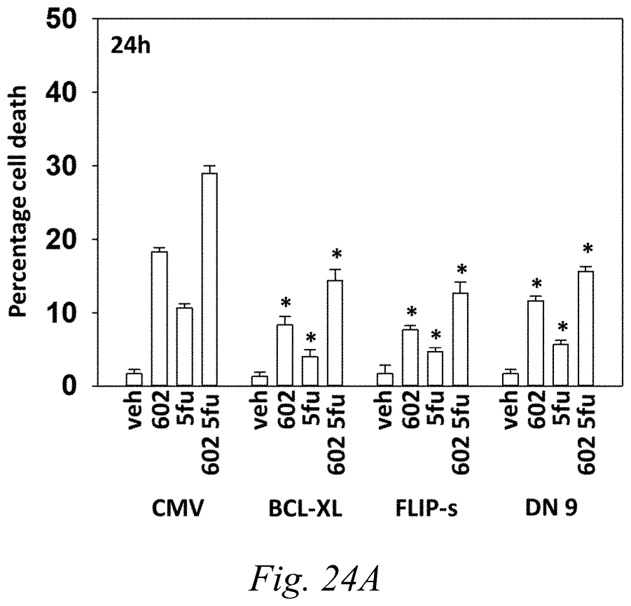

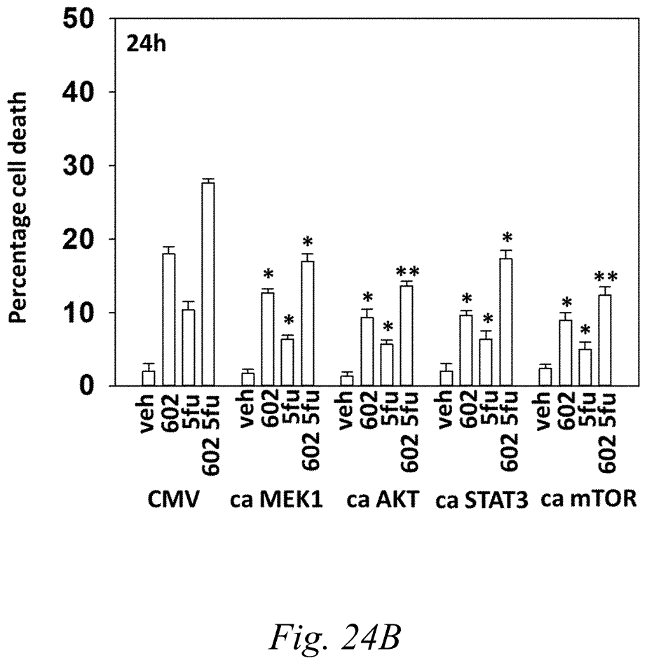

Recent studies with GZ17-6.02 in GI tumor cells demonstrated that it activated a DNA damage/ATM-AMPK-ULK1-‘autophagy’ pathway to cause tumor cell death. In AK cells, knock down of the autophagy-regulatory proteins ULK1, Beclin1, or ATG5 significantly reduced the lethality of GZ17-6.02 alone or in combination with 5FU (FIG. 23). To a lesser extent than ULK1, Beclin1 or ATG5; knock down of ATM, AMPKα, eIF2α and FADD also significantly reduced the lethality of GZ17-6.02 alone or in combination with 5FU (FIG. 23). In separate studies, AK cells were transfected with plasmids to express cyto-protective proteins or activated forms of protein kinases and transcription factors. Over-expression of the caspase 8 inhibitor c-FLIP-s, the mitochondrial protective protein BCL-XL or dominant negative caspase 9 (DN9) all reduced the lethality of GZ17-6.02 alone or in combination with 5FU (FIG. 24A). Expression of activated AKT or activated mTOR suppressed [GZ17-6.02+5FU] lethality to a significantly greater extent than did expression of activated MEK1 or activated STAT3 (FIG. 24B).

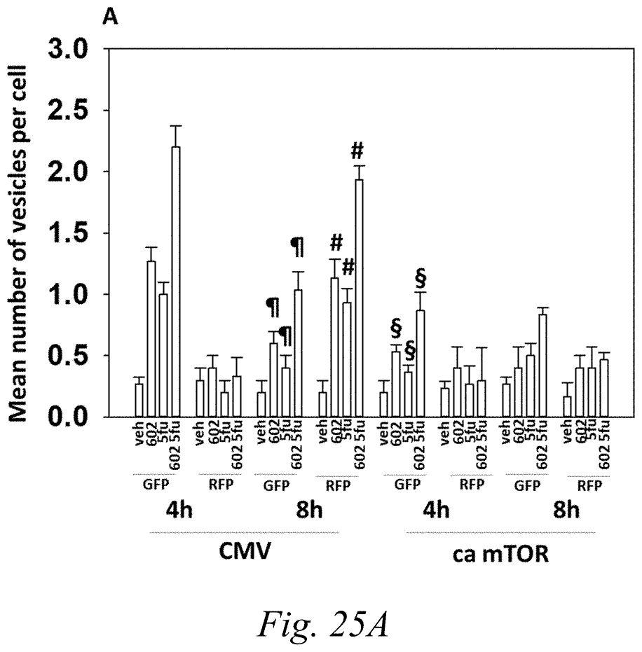

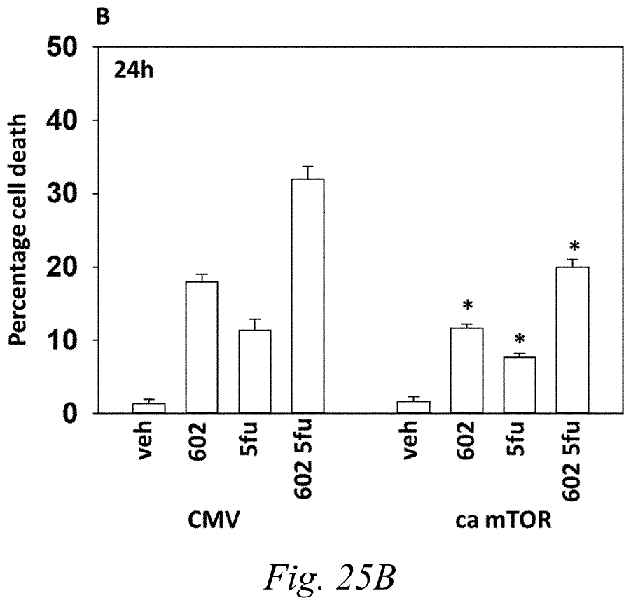

Data from FIGS. 23-24A & B suggested that by blocking autophagy and mitochondrial dysfunction, we largely eliminated GZ17-6.02 toxicity. Loss of mTORC1 activity results in the dephosphorylation of ULK1 S757 which increases ULK1 catalytic activity leading to enhanced autophagosome formation (see also FIG. 4). Expression of an activated mTOR mutant protein suppressed autophagosome formation and prevented the drugs from stimulating autophagic flux (FIG. 25A). Expression of activated mTOR significantly reduced the lethality of the drugs individually and in combination (FIG. 25B). Hence, the data presented above strongly argue that inactivation of mTOR with concomitant activation of ULK1 leading to the formation of autophagosomes is crucial in the primary toxicity of GZ17-6.02 in AK cells, and in the ability of GZ17-6.02 to interact with 5FU to further enhance AK cell killing.

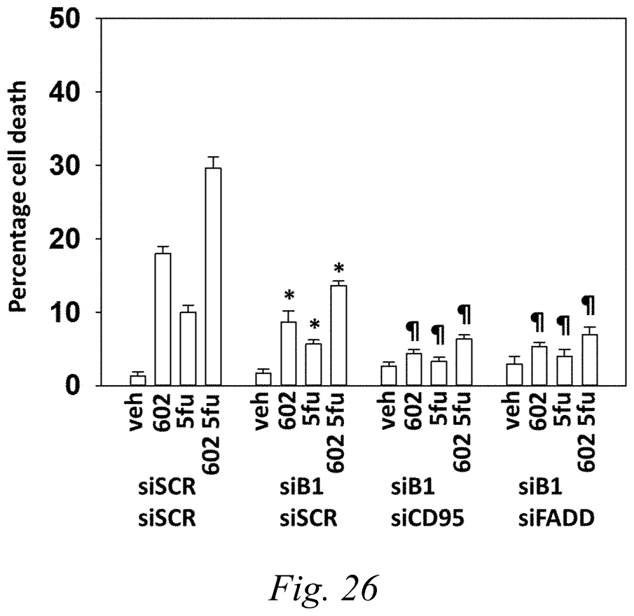

Inhibition of autophagy reduced killing by 50-60% and data in FIG. 24A demonstrated that the caspase 8 inhibitor c-FLIP-s protected the AK cells. This data suggests both autophagy and caspase 8/death receptor signaling played roles in drug-induced killing. In cells lacking Beclin1 expression, tumor cell killing was reduced (FIG. 26). In tumor cells lacking the expression of Beclin1 together with lacking either the death receptor CD95 or the adaptor protein FADD, AK cell killing was abolished. Thus, two coordinated death mechanisms, autophagy and death receptor signaling, play key roles in mediating GZ17-6.02 lethality in AK cells.

Discussion

These experiments were used to define the molecular mechanisms by which GZ17-6.02 killed AK cells. Our data demonstrated that knock down of the essential autophagy-regulatory proteins Beclin1 or ATG5 significantly reduced GZ17-6.02 lethality as a single agent by ˜60% and to a similar degree when it was combined with 5FU. Expression of an activated mutant form of mTOR, which suppressed autophagosome formation, also reduced tumor cell killing by ˜50%. In the absence of autophagy, knock down of the death receptor CD95 or its linker protein FADD, or expression of the caspase 8 inhibitor c-FLIP-s, abolished GZ17-6.02 lethality. Thus, GZ17-6.02 induces two complementary cell killing processes that converge on the mitochondrion to cause caspase-dependent and -independent tumor cell killing.

Multiple alterations in protein expression and cell signaling processes occurred after GZ17-6.02 exposure. The activities of mTORC1 and mTORC2 were reduced and the activity of the AMPK and ULK1 enhanced, which collectively causes autophagosome formation. The expression of ERBB1/3/4 and to a greater extent K-RAS and N-RAS were reduced by GZ17-6.02; surprisingly based on this data, a modest activation of ERK1/2 was observed whereas that of AKT declined. Activation of ERK1/2 was not associated with activation of MEK1/2, implying that inactivation of protein phosphatases which act upon ERK1/2 were inactivated. Expression of an activated MEK1 protein significantly reduced AK cell killing suggesting that the observed ERK1/2 activation was a rapid-response compensatory survival signal. The expression of cytosolic HDAC6 was reduced by GZ17-6.02 and targets of HDAC6, such as the chaperone HSP90 also had their protein levels reduced by the [GZ17-6.02+5FU] combination; this finding may explain why receptor tyrosine kinases chaperoned by HSP90, such as ERBB1, also exhibited reduced expression.

In summary, depending on the target protein and/or pathway, GZ17-6.02 was more effective than the current standard of care (5FU). In most cases, the synergistic combination of GZ17-6.02 and 5FU further increased (or decreased, as applicable) activity to further enhance cell death in the AK cells.

We claim:

1. A medicament for the treatment of actinic keratosis, comprising curcumin, harmine, isovanillin, and fluorouracil.

2. The medicament of claim 1, said curcumin present at a level of from about 5-40% by weight, said harmine present at a level of from about 7-50% by weight, said isovanillin present at a level of from about 25-85% by weight, and said fluorouracil present at a level of from about 0.5-20% by weight, all of the foregoing based upon the total weight of the curcumin, harmine, isovanillin, and fluorouracil taken as 100% by weight.

3. The medicament of claim 1, said curcumin, harmine, isovanillin, and fluorouracil dispersed in a non-interfering solvent.

4. The medicament of claim 1, said solvent selected from the group consisting of C1-C4 alcohols, DMSO, and mixtures thereof.

5. The medicament of claim 1, said medicament designed for topical application on actinic keratosis cells.

6. The medicament of claim 1, each of said curcumin, harmine, and isovanillin being respectively selected from the group consisting of the esters, metal complexes, and pharmaceutically acceptable salts of the curcumin, harmine, and isovanillin, and mixtures thereof.

7. The medicament of claim 1, said curcumin being 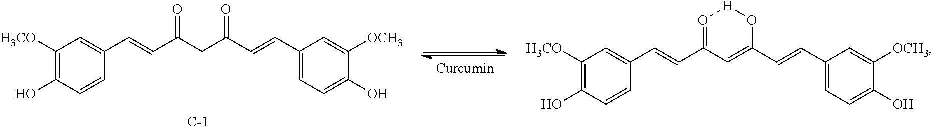
said harmine being 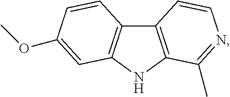 [SMILES: COc1ccc2c(c1)[nH]c1c(C)nccc12]
and said isovanillin being

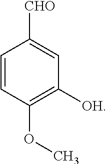

8. A method of treating actinic keratosis, comprising the step of contacting actinic keratosis cells with a medicament, said medicament comprising curcumin, harmine, isovanillin, and fluorouracil.

9. The method of claim 8, said curcumin present at a level of from about 5-40% by weight, said harmine present at a level of from about 7-50% by weight, said isovanillin present at a level of from about 25-85% by weight, and said fluorouracil present at a level of from about 0.5-20% by weight, all of the foregoing based upon the total weight of the curcumin, harmine, isovanillin, and fluorouracil taken as 100% by weight.

10. The method of claim 8, said curcumin, harmine, isovanillin, and fluorouracil dispersed in a non-interfering solvent.

11. The method of claim 8, said solvent selected from the group consisting of C1-C4 alcohols, DMSO, and mixtures thereof.

12. The method of claim 8, including the step of topically applying said medicament onto said actinic keratosis cells.

13. The method of claim 8, each of said curcumin, harmine, and isovanillin being respectively selected from the group consisting of the esters, metal complexes, and pharmaceutically acceptable salts of the curcumin, harmine, and isovanillin, and mixtures thereof.

14. The method of claim 8, said curcumin being 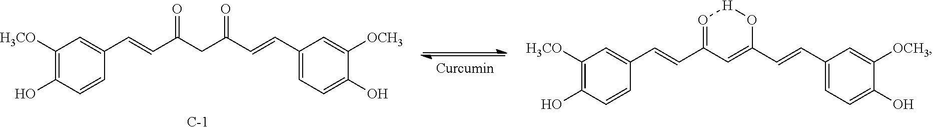
said harmine being 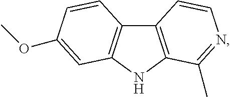 [SMILES: COc1ccc2c(c1)[nH]c1c(C)nccc12]
and said isovanillin being

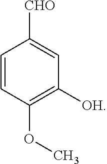

15. A method of treating a human patient suffering from actinic keratosis, comprising the step of administering a medicament to said patient, said medicament comprising curcumin, harmine, isovanillin, and fluorouracil.

16. The method of claim 15, said curcumin present at a level of from about 5-40% by weight, said harmine present at a level of from about 7-50% by weight, said isovanillin present at a level of from about 25-85% by weight, and said fluorouracil present at a level of from about 0.5-20% by weight, all of the foregoing based upon the total weight of the curcumin, harmine, isovanillin, and fluorouracil taken as 100% by weight.

17. The method of claim 15, said curcumin, harmine, isovanillin, and fluorouracil dispersed in a non-interfering solvent.

18. The method of claim 15, said solvent selected from the group consisting of C1-C4 alcohols, DMSO, and mixtures thereof.

19. The method of claim 15, including the step of topically applying said medicament onto said actinic keratosis cells of said human patient.

20. The method of claim 15, each of said curcumin, harmine, and isovanillin being respectively selected from the group consisting of the esters, metal complexes, and pharmaceutically acceptable salts of the curcumin, harmine, and isovanillin, and mixtures thereof.

21. The method of claim 15, said curcumin being 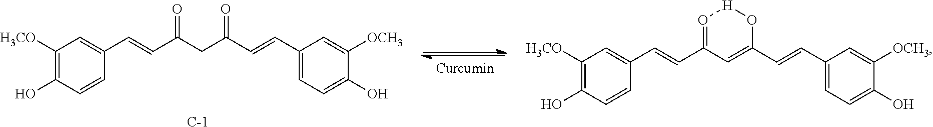
said harmine being 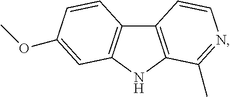 [SMILES: COc1ccc2c(c1)[nH]c1c(C)nccc12]
and said isovanillin being

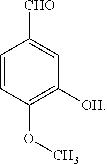

22. A method of treating actinic keratosis, comprising the step of contacting actinic keratosis cells with a medicament, said medicament comprising curcumin, harmine, and isovanillin.

23. The method of claim 22, the ratio of isovanillin:harmine:curcumin in said medicament being approximately 0.1-25:0.1-5:0.1-5.

24. The method of claim 22, said isovanillin being present at a level of from about 25-85% by weight, said harmine being present at a level of from about 7-50% by weight, and said curcumin being present at a level of from about 5-40% by weight, all based on the total weight of the isovanillin, harmine, and curcumin taken as 100% by weight.

25. The method of claim 22, said isovanillin being present at a level of at least about three times greater than each of said harmine and curcumin.

26. The method of claim 22, each of said curcumin, harmine, and isovanillin being respectively selected from the group consisting of the esters, metal complexes, and pharmaceutically acceptable salts of the curcumin, harmine, and isovanillin, and mixtures thereof.

27. The method of claim 22, said curcumin being 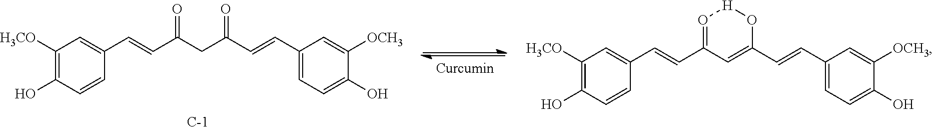
said harmine being 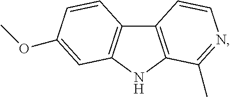 [SMILES: COc1ccc2c(c1)[nH]c1c(C)nccc12]
and said isovanillin being

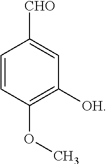

28. A method of treating a human patient suffering from actinic keratosis, comprising the step of administering a medicament to said patient, said medicament comprising curcumin, harmine, and isovanillin.

29. The method of claim 28, the ratio of isovanillin:harmine:curcumin in said medicament being approximately 0.1-25:0.1-5:0.1-5.

30. The method of claim 28, said isovanillin being present at a level of from about 25-85% by weight, said harmine being present at a level of from about 7-50% by weight, and said curcumin being present at a level of from about 5-40% by weight, all based on the total weight of the isovanillin, harmine, and curcumin taken as 100% by weight.

31. The method of claim 28, said isovanillin being present at a level of at least about three times greater than each of said harmine and curcumin.

32. The method of claim 28, each of said curcumin, harmine, and isovanillin being respectively selected from the group consisting of the esters, metal complexes, and pharmaceutically acceptable salts of the curcumin, harmine, and isovanillin, and mixtures thereof.

33. The method of claim 28, said curcumin being 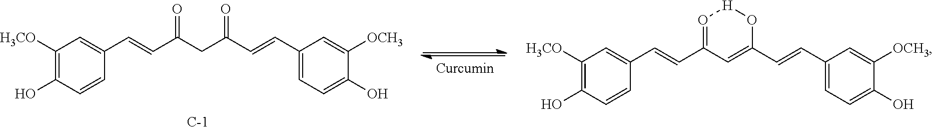
said harmine being 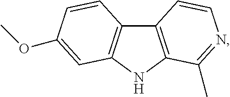 [SMILES: COc1ccc2c(c1)[nH]c1c(C)nccc12]
and said isovanillin being [chemical structure]
